(19) United States
(12) Patent Application Publication
(10) Pub. No.: US 2024/0100710 A1
(43) Pub. Date: Mar. 28, 2024

(54) SYSTEMS AND METHODS FOR ROBOTIC TASTE SENSING

(71) Applicant: Nala Robotics, Inc., Arlington Heights, IL (US)

(72) Inventors: Vijay Kodali, Palatine, IL (US); Ajay Kumar Sunkara, South Barrington, IL (US)

(21) Appl. No.: 17/951,311

(22) Filed: Sep. 23, 2022

(51) Int. Cl. B25J 11/00; B25J 9/16

(52) U.S. Cl. CPC B25J 11/0045; B25J 9/161; B25J 9/1653; B25J 9/1679

(57) ABSTRACT

Systems and methods for a robot kitchen are described. In one embodiment, a computer-implemented method includes identifying a taste profile for a food item prepared by the robot kitchen. The taste profile includes a plurality of predetermined taste parameters. The computer-implemented method also includes generating a taste incidence for the food item by sensing a plurality of sensed taste parameters with a taste sensor module in contact with the food item. The plurality of sensed taste parameters corresponds to the plurality of predetermined taste parameters. The computer-implemented method further includes determining a taste differential by comparing predetermined taste parameters the plurality of predetermined taste parameters to corresponding sensed taste parameters of the plurality of sensed taste parameters. The computer-implemented method also includes calculating feedback for the food item based on the taste differential. The computer-implemented method yet further includes providing the feedback to the robot kitchen.

### BACKGROUND

The Organization for Economic Cooperation and Development found that on average the residents of Western countries spend an average of two hours and eight minutes a day on meal preparation and cleanup. Meal preparation typically includes following a recipe to determine the amounts of ingredients that should be used and how those ingredients should be incorporated during the cooking process. Even slight deviations from the recipe can change the taste of the meal. However, because robots do not have a sense of taste, robots cannot check the prepared meal to ensure that the prepared meal has the desired taste.

### BRIEF DESCRIPTION

According to one aspect, a computer-implemented method for a robot kitchen is provided. The computer-implemented method includes identifying a taste profile for a food item prepared by the robot kitchen. The taste profile includes a plurality of predetermined taste parameters. The computer-implemented method also includes generating a taste incidence for the food item by sensing a plurality of sensed taste parameters with a taste sensor module in contact with the food item. The plurality of sensed taste parameters corresponds to the plurality of predetermined taste parameters. The computer-implemented method further includes determining a taste differential by comparing predetermined taste parameters the plurality of predetermined taste parameters to corresponding sensed taste parameters of the plurality of sensed taste parameters. The computer-implemented method also includes calculating feedback for the food item based on the taste differential. The computer-implemented method yet further includes providing the feedback to the robot kitchen.

According to another aspect, a computer-implemented method for a robot kitchen is provided. The computer-implemented method includes receiving an order for a food item from a user. The user is associated with a user profile including a number of taste preferences. The computer-implemented method includes identifying a recipe for the food item. The recipe includes a taste profile having a plurality of predetermined taste parameters based on the number of taste preferences. The computer-implemented method further includes preparing the food item in the robot kitchen. The computer-implemented method further includes generating a taste incidence for the food item by sensing a plurality of sensed taste parameters with a taste sensor module in contact with the food item. The plurality of sensed taste parameters corresponds to the plurality of predetermined taste parameters. The computer-implemented method yet further includes determining a taste differential by comparing predetermined taste parameters the plurality of predetermined taste parameters to corresponding sensed taste parameters of the plurality of sensed taste parameters. The computer-implemented method includes determining to provide the food item to the user based on the taste differential.

According to still another aspect, a taste sensor module for a robot kitchen is provided. The taste sensor module includes a first sensor probe, a second sensor probe, a taste profile module, a taste incidence module, and an assessment module. The first sensor probe is configured to measure a first sensed taste parameter of a food item when in contact with a food item. The second sensor probe is configured to measure a second sensed taste parameter of a food item when in contact with the food item. The taste profile module is configured to identify a taste profile for the food item prepared by the robot kitchen. The taste profile includes a first predetermined taste parameter and a second predetermined taste parameter. The taste incidence module configured to generate a taste incidence including the first sensed taste parameter and the second sensed taste parameter. The assessment module is configured to determine a taste differential by comparing the first sensed taste parameter to the first predetermined taste parameter and the second sensed taste parameter to the second predetermined taste parameter and calculating feedback for the food item based on the taste differential.

### BRIEF DESCRIPTION OF THE DRAWINGS

The novel features believed to be characteristic of the disclosure are set forth in the appended claims. In the descriptions that follow, like parts are marked throughout the specification and drawings with the same numerals, respectively. The drawing figures are not necessarily drawn to scale and certain figures may be shown in exaggerated or generalized form in the interest of clarity and conciseness. The disclosure itself, however, as well as a preferred mode of use, further objects and advances thereof, will be best understood by reference to the following detailed description of illustrative embodiments when read in conjunction with the accompanying drawings.

FIG. 1 is a block diagram of an operating environment for systems and methods for automated cooking according to an exemplary embodiment.

FIG. 2 is an oblique view of an exemplary robot kitchen for a system for automated cooking, according to one aspect.

FIG. 3 is an exemplary process flow of a method for automated cooking, according to one aspect.

FIG. 4 depicts a partial perspective view of a taste sensor module, according to one aspect.

FIG. 5 is another exemplary process flow of a method for automated cooking, according to one aspect.

FIG. 6 is an exemplary set of instructions for a recipe for automated cooking, according to one aspect.

FIG. 7 is an illustration of an example computer-readable medium or computer-readable device including processor-executable instructions configured to embody one or more of the provisions set forth herein, according to one aspect.

### DETAILED DESCRIPTION

Systems and methods for automated cooking are described herein. As discussed above, a robotic kitchen may be able to prepare a number of food items for human users, but the robotic kitchen is unable taste the prepared food items to determine if the food item is pleasant to taste.

The present systems and methods provide a taste sensor module for automated cooking that allow the robotic kitchen to assess the taste of a prepared food item. In particular, a taste sensor module is a smart and portable device which can sense different taste parameters of the food item that allow the robotic kitchen to assess flavor. For example, the taste sensor module includes one or more probes to sense temperature, salinity and moisture, and uses refractometer principle to sense sweetness, sourness and spice. The sensed taste parameters may be compared to predetermined taste parameters in order to assess the taste of the food. The assessed taste may be framed as a taste differential between the sensed taste parameters and the predetermined taste parameters.

In one embodiment, the taste sensor module may be battery-operated and use a microcontroller-based system with a wireless transceiver to communicate with a central controller. The taste sensor module may also include a display. The display may display the sensed taste parameters for a food item or types of food item. The sensed taste parameters may be displayed accordingly to different categories. For example, the sensed taste parameters may be categorized as high, normal, or low according to different value ranges. For example, suppose a sensed taste parameter is salinity. A predetermined taste parameter for salinity may define a number of value ranges corresponding to high, normal, and low. The display displays “high,” “normal,” or “low” based on the value range that includes the value of the sensed taste parameter for salinity. In this manner, the robot kitchen can use a number of sensed taste parameters to assess the taste of food, and communicate the assessment as feedback.

### Definitions

The following includes definitions of selected terms employed herein. The definitions include various examples and/or forms of components that fall within the scope of a term and that can be used for implementation. The examples are not intended to be limiting. Further, the components discussed herein, can be combined, omitted or organized with other components or into different architectures.

“Bus,” as used herein, refers to an interconnected architecture that is operably connected to other computer components inside a computer or between computers. The bus can transfer data between the computer components. The bus can be a memory bus, a memory processor, a peripheral bus, an external bus, a crossbar switch, and/or a local bus, among others.

“Component,” as used herein, refers to a computer-related entity (e.g., hardware, firmware, instructions in execution, combinations thereof). Computer components may include, for example, a process running on a processor, a processor, an object, an executable, a thread of execution, and a computer. A computer component(s) can reside within a process and/or thread. A computer component can be localized on one computer and/or can be distributed between multiple computers.

“Computer communication,” as used herein, refers to a communication between two or more computing devices (e.g., computer, personal digital assistant, cellular telephone, network device) and can be, for example, a network transfer, a data transfer, a file transfer, an applet transfer, an email, a hypertext transfer protocol (HTTP) transfer, and so on. A computer communication can occur across any type of wired or wireless system and/or network having any type of configuration, for example, a local area network (LAN), a personal area network (PAN), a wireless personal area network (WPAN), a wireless network (WAN), a wide area network (WAN), a metropolitan area network (MAN), a virtual private network (VPN), a cellular network, a token ring network, a point-to-point network, an ad hoc network, a mobile ad hoc network, among others. Computer communication can utilize any type of wired, wireless, or network communication protocol including, but not limited to, Ethernet (e.g., IEEE 802.3), WiFi (e.g., IEEE 802.11), communications access for land mobiles (CALM), WiMax, Bluetooth, Zigbee, ultra-wideband (UWAB), multiple-input and multiple-output (MIMO), telecommunications and/or cellular network communication (e.g., SMS, MMS, 3G, 4G, LTE, 5G, GSM, CDMA, WAVE), satellite, dedicated short range communication (DSRC), among others.

“Computer-readable medium,” as used herein, refers to a non-transitory medium that stores instructions and/or data. A computer-readable medium can take forms, including, but not limited to, non-volatile media, and volatile media. Non-volatile media can include, for example, optical disks, magnetic disks, and so on. Volatile media can include, for example, semiconductor memories, dynamic memory, and so on. Common forms of a computer-readable medium can include, but are not limited to, a floppy disk, a flexible disk, a hard disk, a magnetic tape, other magnetic medium, an ASIC, a CD, other optical medium, a RAM, a ROM, a memory chip or card, a memory stick, and other media from which a computer, a processor or other electronic device can read.

“Database,” as used herein, is used to refer to a table. In other examples, “database” can be used to refer to a set of tables. In still other examples, “database” can refer to a set of data stores and methods for accessing and/or manipulating those data stores. A database can be stored, for example, at a disk and/or a memory.

“Data store,” as used herein can be, for example, a magnetic disk drive, a solid-state disk drive, a floppy disk drive, a tape drive, a Zip drive, a flash memory card, and/or a memory stick. Furthermore, the disk can be a CD-ROM (compact disk ROM), a CD recordable drive (CD-R drive), a CD rewritable drive (CD-RW drive), and/or a digital video ROM drive (DVD ROM). The disk can store an operating system that controls or allocates resources of a computing device.

“Display,” as used herein can include, but is not limited to, LED display panels, LCD display panels, CRT display, plasma display panels, touch screen displays, among others, that are often found on portable devices to display information. The display can receive input (e.g., touch input, keyboard input, input from various other input devices, etc.) from a user.

“Input/output device” (I/O device) as used herein can include devices for receiving input and/or devices for outputting data. The input and/or output can be for controlling different features which include various components, systems, and subsystems. Specifically, the term “input device” includes, but it not limited to: keyboard, microphones, pointing and selection devices, cameras, imaging devices, video cards, displays, push buttons, rotary knobs, and the like. The term “input device” additionally includes graphical input controls that take place within a user interface which can be displayed by various types of mechanisms such as software and hardware-based controls, interfaces, touch screens, touch pads or plug and play devices. An “output device” includes, but is not limited to: display devices, and other devices for outputting information and functions.

“Logic circuitry,” as used herein, includes, but is not limited to, hardware, firmware, a non-transitory computer readable medium that stores instructions, instructions in execution on a machine, and/or to cause (e.g., execute) an action(s) from another logic circuitry, module, method and/or system. Logic circuitry can include and/or be a part of a processor controlled by an algorithm, a discrete logic (e.g., ASIC), an analog circuit, a digital circuit, a programmed logic device, a memory device containing instructions, and so on. Logic can include one or more gates, combinations of gates, or other circuit components. Where multiple logics are described, it can be possible to incorporate the multiple logics into one physical logic. Similarly, where a single logic is described, it can be possible to distribute that single logic between multiple physical logics.

“Memory,” as used herein can include volatile memory and/or nonvolatile memory. Non-volatile memory can include, for example, ROM (read only memory), PROM (programmable read only memory), EPROM (erasable PROM), and EEPROM (electrically erasable PROM). Volatile memory can include, for example, RAM (random access memory), synchronous RAM (SRAM), dynamic RAM (DRAM), synchronous DRAM (SDRAM), double data rate SDRAM (DDRSDRAM), and direct RAM bus RAM (DRRAM). The memory can store an operating system that controls or allocates resources of a computing device.

“Module,” as used herein, includes, but is not limited to, non-transitory computer readable medium that stores instructions, instructions in execution on a machine, hardware, firmware, software in execution on a machine, and/or combinations of each to perform a function(s) or an action(s), and/or to cause a function or action from another module, method, and/or system. A module can also include logic, a software-controlled microprocessor, a discrete logic circuit, an analog circuit, a digital circuit, a programmed logic device, a memory device containing executing instructions, logic gates, a combination of gates, and/or other circuit components. Multiple modules can be combined into one module and single modules can be distributed among multiple modules.

“Operable connection,” or a connection by which entities are “operably connected,” is one in which signals, physical communications, and/or logical communications can be sent and/or received. An operable connection can include a wireless interface, a physical interface, an optical interface, a data interface, and/or an electrical interface.

“Portable device,” as used herein, is a computing device typically capable of computer communication. The portable device may have a display screen with user input (e.g., touch, keyboard) and a processor for computing. Portable devices include, but are not limited to, handheld devices, mobile devices, smart phones, laptops, tablets and e-readers. In some embodiments, a “portable device” could refer to a remote device that includes a processor for computing and/or a communication interface for receiving and transmitting data remotely. In other embodiments, the portable device may be a device for facilitating remote communication. For example, the portable device may be a key fob that remotely controls the security system including the door locks, alarms, etc.

“Processor,” as used herein, processes signals and performs general computing and arithmetic functions. Signals processed by the processor can include digital signals, data signals, computer instructions, processor instructions, messages, a bit, a bit stream, that can be received, transmitted and/or detected. Generally, the processor can be a variety of various processors including multiple single and multicore processors and co-processors and other multiple single and multicore processor and co-processor architectures. The processor can include logic circuitry, such as a programmable logic controller, to execute actions and/or algorithms.

“Robotic system,” as used herein can include, but is not limited to, any automatic or manual systems that can be used to enhance the cooking process. Exemplary robotic systems include, but are not limited to: an electronic mobility and stability control systems, visual devices (e.g., camera systems, proximity sensor systems), a temperature control system, a lighting system, an audio system, and a sensory system, among others.

“User,” as used herein can include, but is not limited to, one or more biological beings. The user can be a human (e.g., an adult, a child, an infant) or an animal (e.g., a pet, a dog, a cat).

A “value” and “level”, as used herein may include, but is not limited to, a numerical or other kind of value or level such as a percentage, a non-numerical value, a discrete state, a discrete value, a continuous value, among others. The term “value of X” or “level of X” as used throughout this detailed description and in the claims refers to any numerical or other kind of value for distinguishing between two or more states of X. For example, in some cases, the value or level of X may be given as a percentage between 0% and 100%. In other cases, the value or level of X could be a value in the range between 1 and 10. In still other cases, the value or level of X may not be a numerical value, but could be associated with a given discrete state, such as “not X”, “slightly x”, “x”, “very x” and “extremely x”.

### I. System Overview

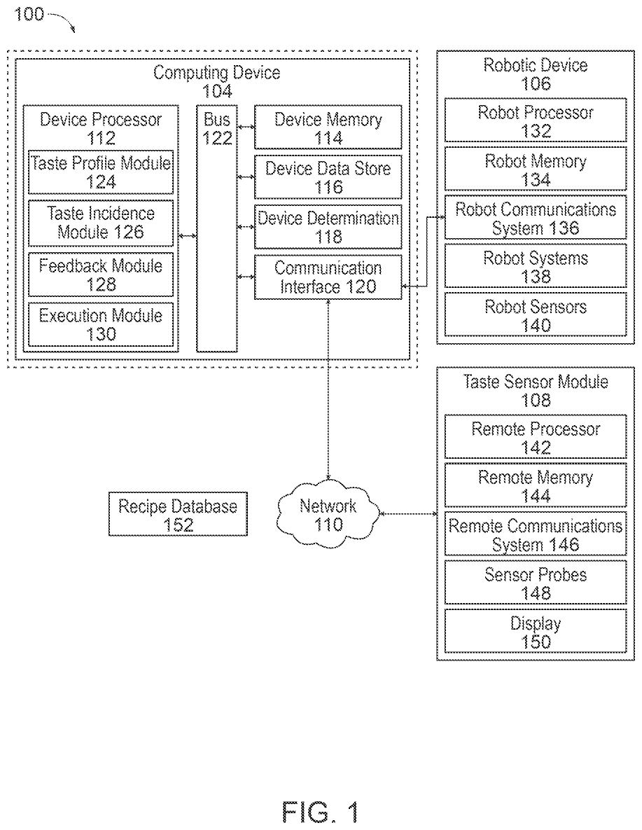

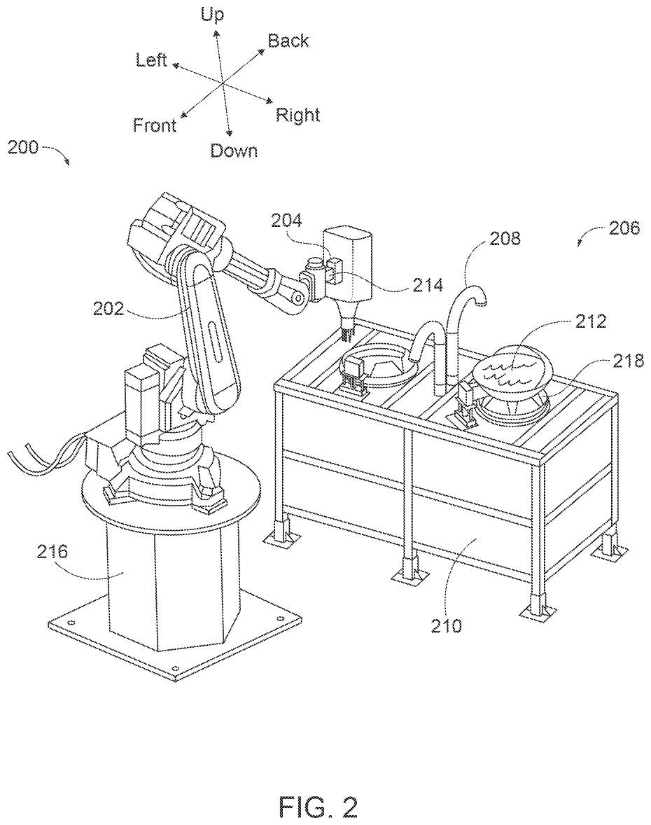

Referring now to the drawings, wherein the showings are for purposes of illustrating one or more exemplary embodiments and not for purposes of limiting same, FIG. 1 is a schematic diagram of an operating environment 100 according to an exemplary embodiment is shown. One or more of the components of the operating environment 100 can be utilized, in whole or in part, with an automated robot kitchen 200, as shown in FIG. 2 or a taste sensor module 108. The components of the operating environment 100, as well as the components of other systems, hardware architectures, and software architectures discussed herein, may be combined, omitted, or organized into different architectures for various embodiments. The operating environment 100 may be implemented with a device or remotely stored.

The operating environment 100 having a computing device 104, a robotic device 106, and the taste sensor module 108 that communicates via a network 110. Generally, the computing device 104 includes a device processor 112, a device memory 114, a device data store 116, a position determination unit 118, and a communication interface 120, which are each operably connected for computer communication via a bus 122 and/or other wired and wireless technologies defined herein. The computing device 104, can include provisions for processing, communicating, and interacting with various components of the operating environment 100. In one embodiment, the computing device 104 can be implemented with the robotic device 106, for example, as part of a telematics unit, a head unit, an electronic control unit, an on-board unit, or as part of a specific robotic system, among others. In other embodiments, the computing device 104 can be implemented remotely, for example, with a portable device (not shown) connected via the network 110.

The device processor 112 can include logic circuitry with hardware, firmware, and software architecture frameworks for automated cooking. Thus, in some embodiments, the device processor 112 can store application frameworks, kernels, libraries, drivers, application program interfaces, among others, to execute and control hardware and functions discussed herein. For example, the device processor 112 can include a taste profile module 124, a taste incidence module 126, and a feedback module 128, and an execution module 130, although it is understood that the device processor 112 can be configured into other architectures. Further, in some embodiments, the device memory 114 and/or the device data store 116 can store similar components as the device processor 112 for execution by the device processor 112.

The modules of the device processor 112 may access the position determination unit 118 via the bus 122. The position determination unit 118 can include hardware (e.g., sensors) and software to determine and/or acquire position data about various components of the operating environment 100, such as the robotic device 106. For example, the position determination unit 118 can include a positioning system (not shown) and/or an inertial measurement unit (IMU) (not shown). Further, the position determination unit 118 can provide dead-reckoning data or motion data from, for example, a gyroscope, accelerometer, magnetometers, among other sensors, such as the robotic sensors 140.

The communication interface 120 can include software and hardware to facilitate data input and output between the components of the computing device 104 and other components of the operating environment 100. Specifically, the communication interface 120 can include network interface controllers (not shown) and other hardware and software that manages and/or monitors connections and controls bi-directional data transfer between the communication interface 120 and other components of the operating environment 100 using, for example, the network 110.

The robotic device 106 include a robot processor 132, a robot memory 134, a robot communications system 136, robotic systems 138, and robotic sensors 140. For example, the robotic device 106 may include the robot kitchen 200 in whole or in part, such as robotic arms 202, end effectuators 204, a cooking surface 206, a liquid dispenser 208, and/or a storage unit 210, among others. For example, the robot processor 132, a robot memory 134, and a robot communications system 136 may be situated in the robotic arms 202 or distributed among multiple components of the robot kitchen 200.

The robotic systems 138 can include any type of robotic control system and/or component of the robotic kitchen 200 described herein to enhance the robotic device 106 for automated cooking one or more food items without human intervention. For example, the robotic systems 138 can include measuring systems, electronic mobility control, electronic stability control, etc. As will be described, one or more of the robotic systems 138 can be controlled remotely according the systems and methods discussed herein.

The robotic sensors 140, which can be implemented with the robotic systems 138, can include various types of sensors for use with the robotic device 106 and/or the robotic systems 138 for detecting and/or sensing a parameter of the associated with automated cooking. For example, the robotic sensors 140 can provide data about ingredients, cooking, recipes, tasks, and/or various components of the operating environment 100. The robotic sensors 140 may include, but are not limited to: acceleration sensors, speed sensors, braking sensors, proximity sensors, and vision sensors, among others. Accordingly, the robotic sensors 140 can be any type of sensor, for example, acoustic, electric, environmental, optical, imaging, light, pressure, force, thermal, temperature, and/or proximity, among others.

The taste sensor module 108 may include a remote processor 142, a remote memory 144, a remote communication system 146, sensor probes 148, and a display 150 that are configured to be in communication with one another. In some embodiments, the taste sensor module 108 can receive and transmit information to and from the computing device 104 and/or the robotic device 106 including, but not limited to recipe information, ingredient data, sensor data, and/or feedback data, etc. In some embodiments, the taste sensor module 108 may operate in conjunction with the operating environment 100, such a third-party recipe aggregators, social media outlets, etc.

Using the system and network configuration discussed above, the robotic devices of the robot kitchen 200 can be controlled to perform automated cooking tasks without human intervention. Detailed embodiments describing exemplary methods using the system and network configuration discussed above will now be discussed in detail.

### II. Methods for Automated Cooking

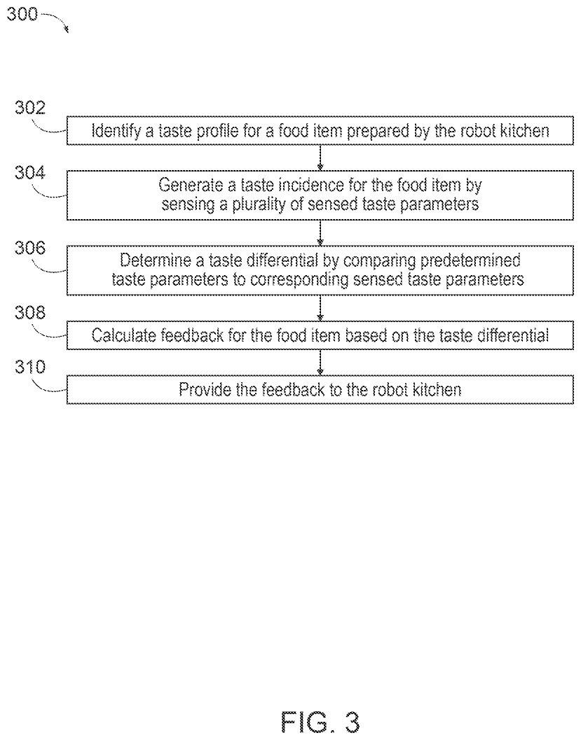

Referring now to FIG. 3, a method 300 for automated cooking will now be described according to an exemplary embodiment. FIG. 3 will also be described with reference to FIGS. 1, 2, and 4-7. For simplicity, the method 300 will be described by the following steps, but it is understood that the elements of the method 300 can be organized into different architectures, blocks, stages, and/or processes.

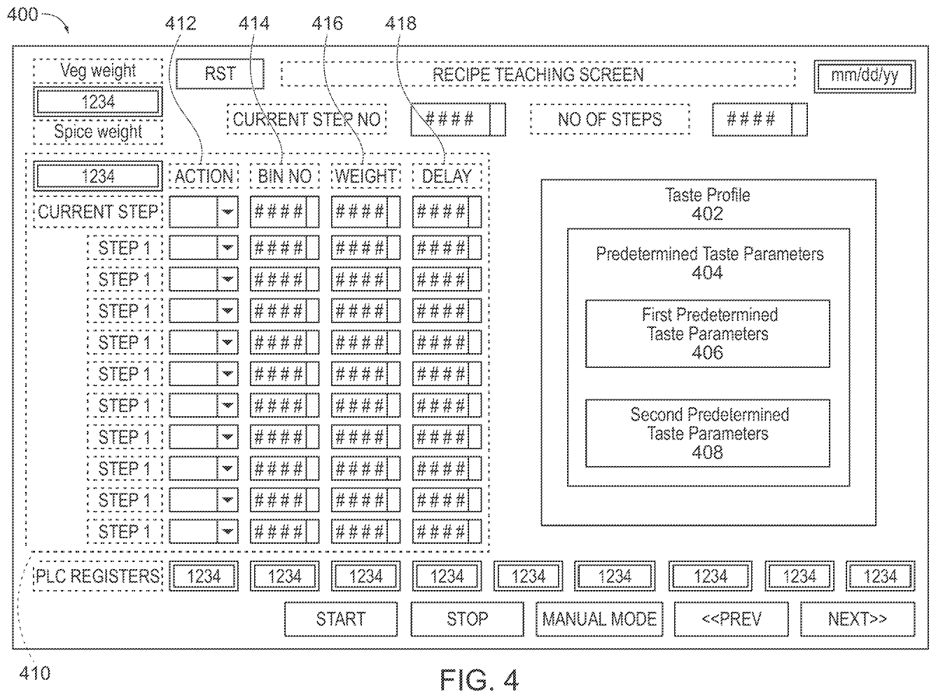

At block 302 the method 300 includes the taste profile module 124 identifying a taste profile 402 for a food item 212 prepared by the robot kitchen 200. The taste profile 402, as shown in FIG. 4, may be associated with the recipe 400 associated with the food item 212. The taste profile 402 includes a plurality of predetermined taste parameters 404 that are values which quantify aspects of flavor and the experience of taste for the food item 212. For example, the predetermined taste parameters 404 may include temperature, salinity, moisture, sweetness, sourness, and spice among others. The predetermined taste parameters 404 may include a value or a range of values. Suppose that the food item 212 is a soup, the predetermined taste parameter for salinity may include a range of 600 part per million (ppm) to 4,000 ppm as the range of values.

In some embodiments, the predetermined taste parameters may include a number of categories. Continuing the example from above, the predetermined taste parameter for salinity may categorize a range of 600 part per million (ppm) to 4,000 ppm as “normal.” Additionally, a range of values from 0 ppm to 600 ppm is categorized as “low” and 4,000 ppm to 10,000 ppm as “high.” Furthermore, the predetermined taste parameter may be a parameter threshold value. For example, below 600 ppm may be categorized as low for salinity of the food item 212 and above 4,000 may be categorized as high for salinity of the food item 212. Accordingly, salinity may have a predetermined taste parameter that defines low, normal, and high.

As described above, the taste profile 402 may include a number of predetermined taste parameters. For example, the predetermined taste parameters may include a first predetermined taste parameter 406, such as the predetermined taste parameter for salinity, described above, and a second predetermined taste parameter 408. The second predetermined taste parameter 408 is associated with a different aspect of the food item 212, and therefore, has different values and/or ranges of values. Suppose that the second predetermined taste parameter 408 for the food item 212, soup, is temperature. Then the range of values between 88° C. and 93° C. may be defined as normal, while lower than 88° C. is categorized as “low” and higher than 88° C. is categorized as “high.” Therefore, the different predetermined taste parameters are associated with different values and ranges for the food item 212. The values and ranges of values are based on aspect of the flavor or taste experience of the food item 212.

In some embodiments, taste profiles for a food item 212 may be based on a type of food item 212. For example, the values and ranges of values may be based on a type of food item 212. For example, suppose there are a number of types of soups on a menu, rather than having a taste profile 402 for each individual soup, the soups may be delineated based on types, such as hot clear soup, hot thick soup, and cold soup. Soups that are defined by the type hot clear soups may each use a taste profile 402 with the predetermined taste parameters 404 tailored to that type of soup. For example, continuing the example from above, the second predetermined taste parameter 408 for the taste profile 402 associated with the type: hot clear soups, may have a value of 99° C., the type hot thick soups may have a range of values for the second predetermined taste parameter 408 as 88° C. and 93° C., and the type cold soups may have a parameter threshold value for the second predetermined taste parameter 408 as 4° C. or colder. Accordingly, the taste profile 402 may be based on the food item 212 or on a type of food item 212.

The taste profile 402 may additionally be based on a user profile. The taste profile module 124 may store a user profile for a user that includes taste profiles and/or preferences for food item 212s and/or types of food items based on preferences of the user. For example, the taste profile module 124 may receive a user profile that indicates that the user prefers low sodium food items. The existing profile for a food item 212 may be adjusted or replaced based on the user profile. Continuing the example from above, suppose that the user has indicated low sodium for soup, the taste profile module 124 may adjust the predetermined taste parameter for salinity from 600 ppm to 4,000 ppm to a 600 ppm to 800 ppm in response to receiving the user profile. In another embodiment, the taste profile module 124 may maintain a default taste profile, and in response to receiving the user profile, the taste profile module 124 may replace the taste profile 402 with a user taste profile from the user profile for the food item 212 or the type of food item. Accordingly, the robotic kitchen can modify the predetermined taste parameters to conform with the preferences with the user such that the robotic kitchen 200 approximates the sense of taste of the user.

At block 304 the method 300 includes the taste incidence module 126 generating a taste incidence 510 for the food item 212 by sensing a plurality of sensed taste parameters 512 with a taste sensor module 108 in contact with the food item 212. The plurality of sensed taste parameters 512 in the taste incidence 510 may correspond to the plurality of predetermined taste parameters 404 of the taste profile 402 for the food item 212. For example, if the predetermined taste parameters 404 include temperature, salinity, moisture, sweetness, sourness, and spice, then the sensed taste parameters 512 also include temperature, salinity, moisture, sweetness, sourness, and spice.

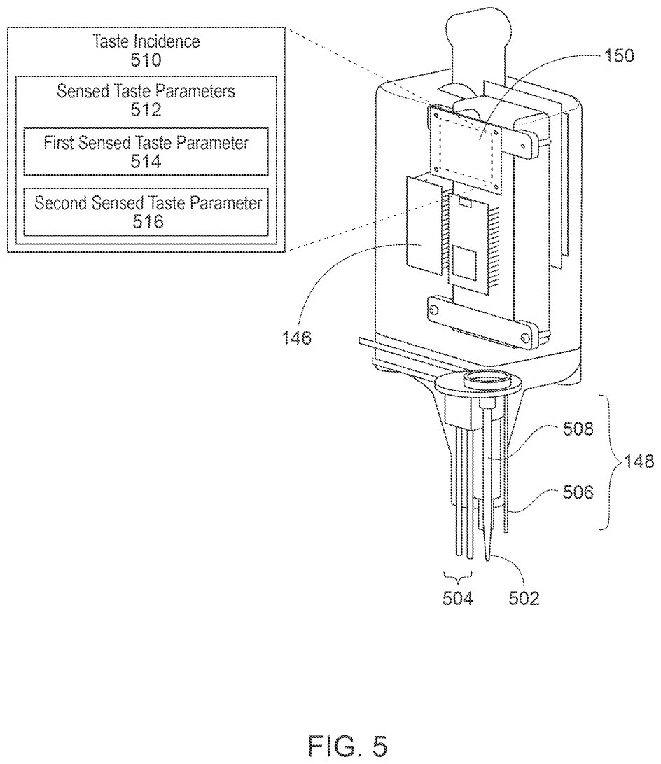

In one embodiment, the taste incidence module 126 generates the taste incidence 510 in response to receiving the plurality of sensed taste parameters 512 from the taste sensor module 108. Turning to FIG. 5, the taste sensor module 108 may include a number of sensor probes 148 such as a temperature sensor 502, a salinity sensor 504, a moisture sensor 506, and a refractometer 508. In one embodiment, the temperature sensor 502 may be a thermistor for measuring temperatures −50° C. to 300° C. The salinity sensor 504 may be a total dissolved solids measurement or water conductivity sensor to measure the conductivity of dissolved ionized solids in the water. The moisture sensor 506 may be a resistive type probe that measures the resistance or impedance. The refractometer 508 may be a hydrometer designed for measuring sugar (i.e., sweetness) or acidity (i.e., sourness). The taste sensor module 108 may be supported on an end effector 214 on a robot arm supported on a base 216 in the robot kitchen 200, defining a reaching distance from the end effector 214 to the base 216. The robot arm may move the taste sensor module 108 around the robot kitchen 200 and bring the taste sensor module 108 into contact with the food item 212.

The sensor probes 148 measure a value for the sensed taste parameters 512 such as a first sensed taste parameter 514 and a second sensed taste parameter 516. For example, when in contact with the food item 212, the taste sensor module 108 measures a sensed taste parameter for the temperature. The taste sensor module 108 is in contact with the food item 212 when one or more of the sensor probes 148 are at least partially in contact with the food item 212. For example, the taste sensor module 108 may be in contact with the food item 212 when one or more of the sensor probes 148 is partially submerged in the food item 212.

At block 306 the method 300 includes the feedback module 128 determining a taste differential by comparing predetermined taste parameters 404 of the plurality of predetermined taste parameters to corresponding sensed taste parameters 512 of the plurality of sensed taste parameters. Continuing the example from above, suppose that the taste profile module 124 identifies a first predetermined taste parameter 406 for salinity such that a range of 600 ppm to 4,000 ppm as “normal,” a range of values from 0 ppm to 600 ppm as “low,” and a range of 4,000 ppm to 10,000 ppm as “high” for the food item 212. Likewise, the taste profile module 124 may identify the second predetermined taste parameter 408 for temperature such that lower than 88° C. is categorized as “low” and higher than 88° C. is categorized as “high” for the food item 212. Furthering the example, suppose that the taste incidence module 126 generates a taste incidence 510 for the food item 212 with a first sensed taste parameter 514 for salinity as 1,200 ppm and a second sensed taste parameter 516 for temperature of 92° C.

The feedback module 128 determines at least one taste differential by comparing the sensed taste parameter to the corresponding predetermined taste parameter. For salinity, the feedback module 128 compares the first sensed taste parameter 514 for salinity of 1,200 ppm to the first predetermined taste parameter 406. Specifically, whether the first sensed taste parameter 514 for salinity of 1,200 ppm falls within the “normal” range of 600 ppm to 4,000 ppm. The feedback module 128 determines a taste differential for each set of corresponding taste parameters. For example, if a predetermined taste parameter for a given taste parameter, such as salinity, is present in the taste profile, and a sensed taste parameter is available in the sensed taste parameter, then the feedback module 128 determines a taste differential for that taste parameter. Here the feedback module 128 may determine that a second predetermined taste parameter 408 and a second sensed taste parameter 516 are given for temperature. Accordingly, the feedback module 128 also determines that the second sensed taste parameter 516 for temperature of 92° C. is greater than the parameter threshold value of 88° C. and is, thus, categorized as “high” for the food item 212. Therefore, the feedback module 128 may calculate a number of differentials for any number of taste parameters.

At block 308 the method 300 includes calculating feedback for the food item based on the at least one taste differential. The feedback may include each of the taste differentials individually. For example, the feedback includes both normal for the taste parameter of salinity and high for the taste parameter of temperature corresponding to the first sensed taste parameter 514 and the second sensed taste parameter 516 respectively.

At block 310 the method 300 includes providing the feedback to the robot kitchen 200. In one embodiment, the feedback module 128 may control the taste sensor module 108 to provide an audio cue indicative of the feedback. The content of the audio cue may be controlled based on volume level, bass level, and pitch, among other audio characteristics based on the feedback. In another embodiment, the feedback module 128 may control the display 150 of the taste sensor module 108 to provide a visual cue of the feedback. The content of the visual cue may be controlled to have a size, hue, and brightness, among other visual characteristics based on the feedback. The audio cue and/or the visual cue may be controlled according to the location of the one or more users. For example, the feedback may be delivered to the portable device of a user.

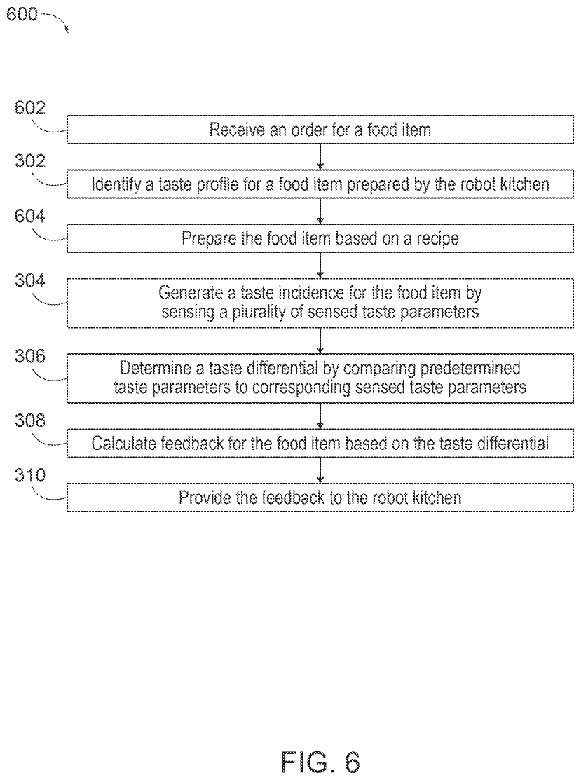

Referring now to FIG. 6, a method 600 for automated cooking will now be described according to an exemplary embodiment. FIG. 6 will also be described with reference to FIGS. 1-5, and 7. For simplicity, the method 600 will be described by the following steps, but it is understood that the elements of the method 600 can be organized into different architectures, blocks, stages, and/or processes.

At block 602 the method 600 includes the taste profile module 124 identifying a recipe 400, as shown in FIG. 4, associated with an order. The order identifies at least one food item to be prepared, served, and/or delivered by the robot kitchen 200. The order may be received from a user. A user may submit the order remotely from or on-site at the robot kitchen 200. An on-site order may be received at the robot kitchen 200. For example, the robot kitchen 200 may be associated with a restaurant or storefront. In another embodiment, the robot kitchen 200 may be located in a kiosk or vending system. Accordingly, an order may be made at the premises of the robot kitchen 200, for example, via a digital menu.

A remote order may be received by the robot kitchen 200 regardless of the location of the user. For example, the order may be received from a portable device via a website or application. In some embodiments, the order may include location services data to identify the robot kitchen 200 closest to the user. Suppose the portable device is a smart phone, the user may order via an application. The order process may include selecting a robot kitchen 200, for example, from a drop-down menu, a map, or a list of robot kitchens that include a selected food item on a menu, etc. As another embodiment, the user may be prompted to order based on an order history. For example, suppose the user routinely orders a curry dish from the robot kitchen on Friday evenings. The user may be prompted to order again via the portable device.

The food item 212 may be selected from a plurality of food items. For example, a menu of the plurality of food items may be populated based on the ingredients in stock at the robot kitchen 200. In another embodiment, the food item 212 may be selected by virtue of a combination of food items being selected. Suppose that a meal includes a first combination of food items: hamburger, French fries, and a milk shake. By selecting the first combination, the order includes at least three food items: hamburger, French fries, and a milk shake. The food item 212 may be offered via a social media, media outlet, or locale. For example, the food item 212 may be associated with a social media personality, such as a celebrity. When the user accesses the social media of the social media personality, the user may be prompted to select the at least one food item.

At least one recipe is identified for the order based on the food items included in the order. Continuing the example from above, suppose that the first combination is selected, recipes for the hamburger, French fries, and a milk shake are identified for the order. The recipes may be stored and identified in the device memory 114, the device data store 116, or the remote memory 144. In one embodiment, the recipes may be stored locally or remotely in a recipe database 152. Receiving the order may cause the taste profile module 124 to query the computing device 104 or recipe database 152 for the one or more recipes associated with the food items included in the order. In one embodiment, the computing device 104 is local to the robotic device 106 and the recipe 400 is received via the network 110.

The recipe 400 includes a set of instructions for preparing the at least one food item. Turning to FIG. 4, a recipe 400 includes an exemplary set of instructions 410 for the recipe 400 for automated cooking, according to one aspect. The set of instructions 410 includes a number of steps. The steps may include actions 412. The actions 412 include operations to be taken by the robot kitchen 200 to facilitate preparation of the food item, for example, stove ignition, preheating, flame control, ingredient identification, ingredient collection instructions, utensil selection, and cooking manipulations (e.g., mix, fold, pour, flip, etc.), among others. The steps may further include location of objects such as ingredients, containers, cookware, utensils, etc.

In one embodiment, the set of instructions may also include locations 414 for compartments of the storage unit 210, such as a bin number. The storage unit 210 may be separated into sections based on the type of ingredient (e.g., spice, vegetable, fruit, meat, dairy, frozen, etc.). The robot kitchen 200 may learn the locations 414 of ingredients based on position values of the compartments and relative distance values to other compartments of the storage unit 210. The set of instructions may further include weight 416, cooking times 418, serving instruction, and flame temperature, among others. Therefore, the recipe 400 may include details for the food item 212, such as the ingredients for the food item, a quantity (e.g., number, weight, volume, etc.) of each ingredient, a sequence of operations to be performed by the robot kitchen 200, necessary utensils and/or cookware, and any related activities to prepare the food item 212. In some embodiments, the steps may include multiple actions and therefore, multiple locations 414, weight 416, cooking times 418, etc.

At block 302 the method 600 includes the taste profile module 124 identifying a taste profile 402 for a food item 212 prepared by the robot kitchen 200. The taste profile module 124 may identify the taste profile 402 in the recipe 400. As discussed above, the taste profile 402 includes the predetermined taste parameters.

At block 604 the method 600 includes the execution module 130 executing the recipe 400 to create the food item 212. Because the operations are performed by the robot kitchen 200, the set of instructions 410 are directed to robotic actions that include precise motions, timing data, etc. For example, the robot kitchen 200 may be mapped to a coordinate system. The set of instructions 410 may include precise coordinates or differential coordinates that dictate movements of the robot kitchen 200. The set of instructions 410 allow the mechanisms of the robot kitchen 200, such as the robotic arms 202 to track the precise location, transport, and/or manipulate the other mechanisms, ingredients, cookware, utensils, etc. Continuing the example from above, a task may include stirring the beaten eggs in the cookware 218 for a predetermined amount of time until a desired consistency is reached. In this manner, the robot kitchen 200 prepares the food item 212.

At block 304 the method 600 includes the taste incidence module 126 generating a taste incidence 510 for the food item 212 by sensing a plurality of sensed taste parameters 512 with a taste sensor module 108 in contact with the food item 212, as described above with respect to method 300. For example, the taste sensor module 108 may at least partially submerged in the food item 212 to measure the sensed taste parameters 512. The sensed taste parameters 512 may be stored in the remote memory 144 of the taste sensor module 108 or the device memory 114 of the computing device 104. Additionally or alternatively, the taste incidence module 126 may receive the sensed taste parameters 512 via the network 110.

At block 306 the method 600 includes the feedback module 128 determining a taste differential by comparing predetermined taste parameters 404 of the plurality of predetermined taste parameters to corresponding sensed taste parameters 512 of the plurality of sensed taste parameters 512, in the manner described with respect to the method 300. At block 308 the method 600 includes calculating feedback for the food item based on the at least one taste differential, in the manner described with respect to the method 300.

At block 310 the method 600 includes providing the feedback to the robot kitchen. In one embodiment, the feedback module 128 may control the taste sensor module 108 to provide an audio cue and/or visual cue indicative of the feedback, in the manner described with respect to the method 300.

In some embodiments, the feedback may include a number of revisions to the set of instructions 410 of the recipe 400. For example, recipe 400 may include changes to the food item 212, such as the ingredients for the food item, a quantity (e.g., number, weight, volume, etc.) of each ingredient, a sequence of operations to be performed by the robot kitchen 200, necessary utensils and/or cookware, and any related activities to prepare the food item 212. The steps of the set of instructions 410 may be changed such that the multiple locations 414, weight 416, cooking times 418, etc. are changed based on the one or more taste differentials.

The ingredients of the set of instructions 410 may be changed based on the taste differential. Suppose that the taste parameter for salinity is determined to be high based on the taste differential. The feedback may include reducing the amount of an ingredient (e.g., salt, broth, etc.). The amount of the ingredient may be reduced by a predetermined increment when a category of the predetermined taste parameter is satisfied. For example, if the category is determined to be high or low, as described above, the amount of the ingredient may be changed by the taste differential. In another embodiment, the amount of change may be based on the taste differential or the sensed taste parameter. For example, the larger the taste differential, the larger the amount of change. If the salt added to a recipe is one teaspoon but the taste differential is greater than a first parameter threshold value (e.g., 4,000 ppm), then the salt added to the in the set of instructions 410 may be reduced to ¾ of a teaspoon, whereas if the taste differential is greater than a second parameter threshold value (e.g., 6,000 ppm), then the salt added in the set of instructions 410 may be reduced to ½ of a teaspoon. In this manner, the change to the recipe is dependent on the taste differential.

In some embodiments, the steps may include multiple actions and therefore, the feedback may alter a plurality of steps of the set of instructions 410. For example, if the taste parameter for temperature of the food item 212 was also listed as high, then the cook time or cook temperature may be altered in one or more of the steps of the set instructions 410 to lower the temperature of the food item 212. This feedback may be included with the alterations to reduce the salinity based on the taste parameter for salinity. Accordingly, the feedback module 128 may alter a number of steps of the set of instructions 410 to address a plurality of taste differentials.

In some embodiments, the feedback module 128 may alter the set of instructions 410 after the robot kitchen 200 prepares the food item 212 a number of time and determines a taste differential a number of times greater than a repetition threshold. The repetition threshold may be set to avoid changing the recipe 400 for a food item 212 every time the food item is prepared. For example, to accommodate for changes in the taste, quality, and age of ingredients, the feedback module 128 may generate feedback that alters the recipe in response to a parameter threshold value being exceeded for the repetition threshold, for example, the food item 212 being prepared on three occasions by the robot kitchen 200. The repetition threshold may include a number of times the food item 212 is prepared, a number of consecutive times the food item 212 is prepared by the robot kitchen, or a number of times the food item is prepared by the robot kitchen 200 and/or a remote robot kitchen.

The repetition threshold may be exceeded when a parameter threshold value is exceeded the number of times defined in the repetition threshold. For example, the feedback may alter the steps of the set of instructions 410 for a taste parameter when a parameter threshold value for that taste parameter is exceeded based on the repetition threshold. Accordingly, the feedback may tune the recipe 400 without making continual changes. In this manner, the robotic kitchen can develop a sense of taste using the taste sensor module 108 and tune the recipes 400.

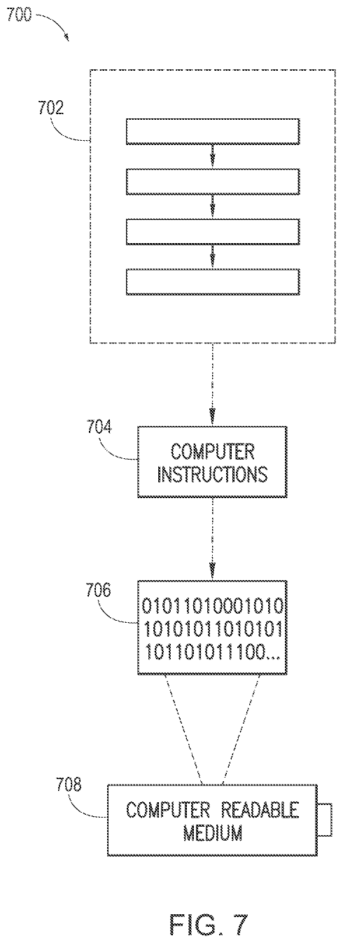

Still another aspect involves a computer-readable medium including processor-executable instructions configured to implement one aspect of the techniques presented herein. An aspect of a computer-readable medium or a computer-readable device devised in these ways is illustrated in FIG. 7, wherein an implementation 700 includes a computer-readable medium 708, such as a CD-R, DVD-R, flash drive, a platter of a hard disk drive, etc., on which is encoded computer-readable data 706. This encoded computer-readable data 706, such as binary data including a plurality of zero's and one's as shown in 706, in turn includes a set of processor-executable computer instructions 704 configured to operate according to one or more of the principles set forth herein. In this implementation 700, the processor-executable computer instructions 704 may be configured to perform a method 702, such as the method 300 of FIG. 3 and the method 600 of FIG. 6. In another aspect, the processor-executable computer instructions 704 may be configured to implement a system, such as the operating environment 100 of FIG. 1 in the robot kitchen 200 shown in FIG. 2. Many such computer-readable media may be devised by those of ordinary skill in the art that are configured to operate in accordance with the techniques presented herein.

As used in this application, the terms “component”, “module,” “system”, “interface”, and the like are generally intended to refer to a computer-related entity, either hardware, a combination of hardware and software, software, or software in execution. For example, a component may be, but is not limited to being, a process running on a processor, a processing unit, an object, an executable, a thread of execution, a program, or a computer. By way of illustration, both an application running on a controller and the controller may be a component. One or more components residing within a process or thread of execution and a component may be localized on one computer or distributed between two or more computers.

Further, the claimed subject matter is implemented as a method, apparatus, or article of manufacture using standard programming or engineering techniques to produce software, firmware, hardware, or any combination thereof to control a computer to implement the disclosed subject matter. The term “article of manufacture” as used herein is intended to encompass a computer program accessible from any computer-readable device, carrier, or media. Of course, many modifications may be made to this configuration without departing from the scope or spirit of the claimed subject matter.

Generally, aspects are described in the general context of “computer readable instructions” being executed by one or more computing devices. Computer readable instructions may be distributed via computer readable media as will be discussed below. Computer readable instructions may be implemented as program modules, such as functions, objects, Application Programming Interfaces (APIs), data structures, and the like, that perform one or more tasks or implement one or more abstract data types. Typically, the functionality of the computer readable instructions are combined or distributed as desired in various environments.

The term “computer readable media” includes communication media. Communication media typically embodies computer readable instructions or other data in a “modulated data signal” such as a carrier wave or other transport mechanism and includes any information delivery media. The term “modulated data signal” includes a signal that has one or more of its characteristics set or changed in such a manner as to encode information in the signal.

Although the subject matter has been described in language specific to structural features or methodological acts, it is to be understood that the subject matter of the appended claims is not necessarily limited to the specific features or acts described above. Rather, the specific features and acts described above are disclosed as example aspects. Various operations of aspects are provided herein. The order in which one or more or all of the operations are described should not be construed as to imply that these operations are necessarily order dependent. Alternative ordering will be appreciated based on this description. Further, not all operations may necessarily be present in each aspect provided herein.

As used in this application, “or” is intended to mean an inclusive “or” rather than an exclusive “or”. Further, an inclusive “or” may include any combination thereof (e.g., A, B, or any combination thereof). In addition, “a” and “an” as used in this application are generally construed to mean “one or more” unless specified otherwise or clear from context to be directed to a singular form. Additionally, at least one of A and B and/or the like generally means A or B or both A and B. Further, to the extent that “includes”, “having”, “has”, “with”, or variants thereof are used in either the detailed description or the claims, such terms are intended to be inclusive in a manner similar to the term “comprising”.

Further, unless specified otherwise, “first”, “second”, or the like are not intended to imply a temporal aspect, a spatial aspect, an ordering, etc. Rather, such terms are merely used as identifiers, names, etc. for features, elements, items, etc. For example, a first channel and a second channel generally correspond to channel A and channel B or two different or two identical channels or the same channel. Additionally, “comprising”, “comprises”, “including”, “includes”, or the like generally means comprising or including, but not limited to.

It will be appreciated that several of the above-disclosed and other features and functions, or alternatives or varieties thereof, may be desirably combined into many other different systems or applications. Also, that various presently unforeseen or unanticipated alternatives, modifications, variations or improvements therein may be subsequently made by those skilled in the art which are also intended to be encompassed by the following claims.

1. A computer-implemented method for a robot kitchen, the computer-implemented method comprising:
identifying a taste profile for a food item prepared by the robot kitchen, wherein the taste profile includes a plurality of predetermined taste parameters;
generating a taste incidence for the food item by sensing a plurality of sensed taste parameters with a taste sensor module in contact with the food item, wherein the plurality of sensed taste parameters corresponds to the plurality of predetermined taste parameters;
determining a taste differential by comparing predetermined taste parameters the plurality of predetermined taste parameters to corresponding sensed taste parameters of the plurality of sensed taste parameters; and
calculating feedback for the food item based on the taste differential; and
providing the feedback to the robot kitchen.

2. The computer-implemented method of claim 1, wherein the plurality of predetermined taste parameters are based on a recipe for the food item, and wherein the feedback includes a modification to the recipe.

3. The computer-implemented method of claim 1, wherein the food item is prepared by the robot kitchen in response to receiving an order for the food item from a user, and wherein predetermined taste parameters of the plurality of predetermined taste parameters are each defined as a range of values, and wherein the range of values are based on a user profile of the user.

4. The computer-implemented method of claim 3, wherein the range of values includes a first set of values for a first user and a second set of values for a second user, and wherein the first set of values is different from the second set of values.

5. The computer-implemented method of claim 1, wherein predetermined taste parameters include a predetermined salinity parameter, a predetermined moisture parameter, and a predetermined temperature parameter corresponding to sensed taste parameters including a sensed salinity parameter, a sensed moisture parameter, and a sensed temperature parameter.

6. The computer-implemented method of claim 1, wherein comparing predetermined taste parameters the plurality of predetermined taste parameters to corresponding sensed taste parameters of the plurality of sensed taste parameters includes determining whether a value of a sensed taste parameter is included in a range of values associated with a corresponding predetermined taste parameter.

7. The computer-implemented method of claim 6, wherein the taste differential includes whether a value of a sensed taste parameter satisfies the range of values associated with a corresponding predetermined taste parameter, is above the range of values, or under the range of values.

8. The computer-implemented method of claim 1, wherein providing the feedback includes displaying the feedback.

9. A computer-implemented method for preparing food items in a robot kitchen, the computer-implemented method comprising:
receiving an order for a food item from a user, wherein the user is associated with a user profile including a number of taste preferences;
identifying a recipe for the food item, wherein the recipe includes a taste profile having a plurality of predetermined taste parameters based on the number of taste preferences;
preparing the food item in the robot kitchen;
generating a taste incidence for the food item by sensing a plurality of sensed taste parameters with a taste sensor module in contact with the food item, wherein the plurality of sensed taste parameters corresponds to the plurality of predetermined taste parameters;
determining a taste differential by comparing predetermined taste parameters the plurality of predetermined taste parameters to corresponding sensed taste parameters of the plurality of sensed taste parameters; and
determining to provide the food item to the user based on the taste differential.

10. The computer-implemented method of claim 9, wherein predetermined taste parameters of the plurality of predetermined taste parameters are each defined as a range of values, and wherein the range of values are based on a user profile of the user.

11. The computer-implemented method of claim 10, wherein the range of values includes a first set of values for a first user and a second set of values for a second user, and wherein the first set of values is different from the second set of values.

12. The computer-implemented method of claim 9, wherein predetermined taste parameters include a predetermined salinity parameter, a predetermined moisture parameter, and a predetermined temperature parameter corresponding to sensed taste parameters including a sensed salinity parameter, a sensed moisture parameter, and a sensed temperature parameter.

13. The computer-implemented method of claim 9, wherein comparing predetermined taste parameters the plurality of predetermined taste parameters to corresponding sensed taste parameters of the plurality of sensed taste parameters includes determining whether a value of a sensed taste parameter is included in a range of values associated with a corresponding predetermined taste parameter.

14. The computer-implemented method of claim 13, wherein the taste differential includes whether a value of a sensed taste parameter satisfies the range of values associated with a corresponding predetermined taste parameter, is above the range of values, or under the range of values.

15. The computer-implemented method of claim 9, wherein the user is provided the food item when the taste differential is below a threshold value.

16. A taste sensor module for a robot kitchen, the taste sensor module comprising:
a first sensor probe configured to measure a first sensed taste parameter of a food item when in contact with a food item;
a second sensor probe configured to measure a second sensed taste parameter of a food item when in contact with the food item;
a taste profile module configured to identify a taste profile for the food item prepared by the robot kitchen, wherein the taste profile includes a first predetermined taste parameter and a second predetermined taste parameter;
a taste incidence module configured to generate a taste incidence including the first sensed taste parameter and the second sensed taste parameter; and
an assessment module configured to determine a taste differential by comparing the first sensed taste parameter to the first predetermined taste parameter and the second sensed taste parameter to the second predetermined taste parameter and calculating feedback for the food item based on the taste differential.

17. The taste sensor module of claim 16, wherein the taste profile module is further configured to receive a recipe for the food item, and wherein the first predetermined taste parameter and the second predetermined taste parameter are based on the recipe for the food item.

18. The taste sensor module of claim 16, wherein the food item is prepared by the robot kitchen in response to receiving an order for the food item from a user, and wherein the first predetermined taste parameter and the second predetermined taste parameter are each defined as a range of values, and wherein the range of values are based on a user profile of the user.

19. The taste sensor module of claim 16, wherein comparing the first predetermined taste parameter and the second predetermined taste parameter to the first sensed taste parameter and the second sensed taste parameter includes determining whether a value of the first sensed taste parameter or the second sensed taste parameter is included in a range of values associated with the first predetermined taste parameter and the second predetermined taste parameter.

20. The taste sensor module of claim 16, further comprising a display configured to display the feedback.
